(19) United States
(12) Patent Application Publication
(10) Pub. No.: US 2007/0266450 A1
(43) Pub. Date: Nov. 15, 2007

(54) Non-human animal alzheimer's disease model and uses thereof

(76) Inventors: Ignacio Torres-Aleman, Madrid (ES); Eva Carro, Madrid (ES); Delphine Bohl, Palaiseau (FR); Jean-Michel Heard, Paris (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/798,882

(22) Filed: May 17, 2007

Related U.S. Application Data
(63) Continuation of application No. PCT/EP05/13022, filed on Nov. 18, 2005.

(30) Foreign Application Priority Data
Nov. 18, 2004 (CA) 2,488,113

(51) Int. Cl. A01K 67/027; A61K 48/00; C12N 15/09

(52) U.S. Cl. 800/12; 514/44; 435/455

(57) ABSTRACT

The present invention relates to the field of diseases, such as Alzheimer's disease, where abnormal brain accumulation of β amyloid and/or amyloid plaques are involved. More specifically, the present invention relates to a non-human animal model for such diseases and its use in screening methods for molecules for treating same.

### FIELD OF THE INVENTION

The present invention relates to the field of diseases, such as Alzheimer's disease, where abnormal brain accumulation of β amyloid and/or amyloid plaques are involved. More specifically, the present invention relates to a non-human animal model for such diseases and its use in screening methods for molecules for treating same.

### BRIEF DESCRIPTION OF THE PRIOR ART

Alzheimer's disease (AD) is becoming one of the most frequent diseases in modern societies probably due to a longer life-span brought about by medical and societal advances¹. Studies with familial forms of the disease determined that brain accumulation of amyloid peptides, a hallmark of the disease, is probably the single most important pathogenic event in AD². Despite being the subject of intense scrutiny, the mechanisms underlying abnormal brain accumulation of β amyloid (Aβ) are not yet elucidated. However, the therapeutic benefit of the reduction of amyloid load is now well established³. Preventing brain amyloidosis may therefore lead to erradication of AD, a goal that currently appears unattainable.

There is therefore a need in the art for new tools in the discovery of molecules in the prevention and treatment of diseases, such as Alzheimer's disease, where abnormal brain accumulation of β amyloid and/or amyloid plaques are involved. There is also a need to provide for new screening and treating methods with regards to such diseases.

### SUMMARY

The present invention satisfies at least one of the above-mentioned needs.

More specifically, an object of the invention concerns a non-human animal used as a model for disease where abnormal brain accumulation of β amyloid and/or amyloid plaques are involved, wherein β amyloid clearance from brain is decreased.

Other objects of the invention concern a method for screening a molecule for the treatment of diseases where abnormal brain accumulation of β amyloid and/or amyloid plaques are involved wherein said method comprises administering said molecule to an animal according to the invention during a time and in an amount sufficient for the Alzheimer's disease-like disturbances to revert, wherein reversion of Alzheimer's disease-like disturbances is indicative of a molecule for the treatment of diseases where abnormal brain accumulation of β amyloid and/or amyloid plaques are involved.

The invention also relates to a method for screening a molecule to prevent the disease from occurring, wherein said molecule prevents or postpones Alzheimer's disease-like disturbance.

Still another object of the invention is to provide a method for treating or preventing a disease where abnormal brain accumulation of β amyloid and/or amyloid plaques are involved in a mammal, wherein said method comprises administering to said mammal a molecule capable of increasing β amyloid clearance from brain.

Yet another object of the invention concerns a process for screening an active molecule interacting with the IGF-I receptor which comprises administering said molecule to an animal during a time and in an amount sufficient for Alzheimer's disease-like disturbances to be modulated, wherein reversion of Alzheimer's disease-like disturbances is indicative of a molecule that increases IGF-I receptor activity and wherein appearance of Alzheimer's disease-like disturbances is indicative of a molecule that decreases IGF-I receptor activity.

A further object of the invention concerns gene transfer vectors capable of either expressing a dominant negative IGF-I receptor or a functional IGF-I receptor.

Yet, a further object of the invention concerns the use of the nucleotide sequence encoding the receptor of IGF-I for the treatment of a disease where abnormal brain accumulation of β amyloid and/or amyloid plaques are involved.

### BRIEF DESCRIPTION OF THE FIGURES

FIG. 1: Blockade of IGF-I signaling in the choroid plexus.

a, HIV-mediated expression of a DN-IGF-IR (KR) blocks IGF-I signaling on cultured choroid plexus epithelial cells. Infected cells do not respond to IGF-I as determined by absence of IGF-I-induced phosphorylation of IGF-IR (pTyrIGF-IR, two viral dilutions tested) and of its downstream kinase Akt (pAkt). Total levels of IGF-IR and Akt remained unaltered. Blots representative of 3 experiments are shown. b, Blockade of IGF-IR in choroid plexus cells results in inhibition of IGF-1-induced albumin transcytosis across the cell monolayer. Representative blot and densitometry histograms are shown. n=3; **p<0.01 vs albumin only. c, GFP expression 3 months after a single icv injection of HIV-GFP. Left: low magnification micrograph depicting GFP expression at the injection site including the choroid plexus of the lateral ventricle and periventricular ependyma; Right: higher magnification micrograph to illustrate GFP expression in choroid plexus cells. A representative rat is shown (n=6). CP, choroid plexus, LV, lateral ventricle. d-f, In vivo IGF-IR blockade after icv delivery of HIV-KR abrogates IGF-I signaling on choroid plexus. d, Intracarotid injection of IGF-I to intact rats results in increased pAkt staining in the choroid plexus. Left: photomicrographs showing pAkt staining in choroid plexus epithelial cells of saline injected (left) and IGF-I injected rats (right). Blot: levels of pAkt are increased after IGF-I. This experiment was done in 3 rats. e, Eight weeks after KR-injection, pAkt levels are no longer. increased in the choroid plexus in response to intracarotid IGF-I, as compared to void-vector injected rats (Control). n=3; *p<0.05 vs control+IGF-I. f, On the contrary, the pAkt response to intracerebral IGF-I is preserved after KR administration. pAkt levels were measured in hippocampal tissue surrounding the injection site. Total Akt levels are shown in lower representative blots. n=3; **p<0.01 vs IGF-I-treated groups g, Passage of intracarotid injected digoxigenin-labelled (DIG) IGF-I into the CSF is blocked 8 weeks after icv injection of KR to adult rats. Representative blot and densitometry histograms. n=3; **p<0.01 vs control.

FIG. 2: Alzheimer's-like neuropathology after in vivo blockade of IGF-IR.

a, Western blot analysis with a pan-specific anti-Aβ antibody shows increased Aβ levels in cortex (left) and decreased in CSF (right) after 3 and 6 months of KR injection. Representative blots and densitometry histograms are shown. Controls n=13, three months n=6; six months n=7; *p<0.05 and **p<0.01 vs controls. b, ELISA analysis of cortical tissue of KR-injected rats after 6 months shows increases in Aβ₁₋₄₀, while Aβ₁₋₄₂ remains unchanged. n=7; **p<0.01. c, Parallel decreases in brain (cortex, upper panels) and CSF levels (lower panels) of Aβ carriers such as albumin (left), transthyretin (middle) and apolipoprotein J (apoJ, right) are found 3/6 months after KR. Number of animals as in panel a; *p<0.05 and **p<0.01 vs controls. d, Cognitive deterioration in KR-treated rats is evident at 3 (triangles) and 6 (squares) months after the injection as determined in the water maze test. Both the acquisition (learning) and the retention (memory) phases of the test were affected. *p<0.05 vs KR at 3 and 6 months. Controls (rhombs) n=13; KR three months n=6; six months n=7.

FIG. 3: Alzheimer's-like neuropathology after in vivo blockade of IGF-IR.

a, Levels of dynamin 1 and synaptophysin in cortex are decreased 6 months after KR, while those of GFAP are increased. Representative blots (left) and densitometry histograms (n=6); *p<0.05 and **p<0.01 vs controls. b, Brain levels of pTyr²¹⁶GSK-3β and pSer⁹GSK-3β are oppositely regulated. after 3 months of KR, resulting in an increased ratio of the active form of this tau-kinase. Representative blots and densitometry histograms. N═; *p<0.05 and **p<0.01 vs controls. c, Blockade of IGF-IR in the choroid plexus results in heavy PHF-tau brain immunostaining and significantly higher HPF-tau levels. Left: upper photomicrographs illustrates abundant PHF-tau⁺ (red) neuronal (calbindin⁺, green) profiles in the hippocampus after 6 months of KR injection. Note the sparing of HPF-tau immunostaining in control neurons as well as the presence of occasional extracellular HPF-tau deposits in KR rats. GL, granule cell layer, hi, hylus. Middle: Thioflavin-S staining of human AD brain and KR-injected rat brain show the presence of tangles (asterisk) in human but not rat sections. Lower: PHF-tau immunostaining in KR-injected rats and human AD brain sections revealed with diaminobenzydine illustrate the presence of similar intracellular deposits. Right: levels of PHF-tau are increased in the brain of KR-injected rats 3/6 months later. Representative blots and densitometry analysis. Levels of tau remained unaffected (lower blot). n=6; *p<0.05 and **p<0.01 vs controls. d, left: As determined by confocal analysis, PHF-tau (red) deposits co-localize with ubiquitin (green) and are surrounded (right panels) by abundant astrocytic (GFAP⁺, green) profiles. Note the absence of tauopathy in void vector-injected animals (control). Cortical sections are shown.

FIG. 4: Restoring IGF-IR function in the choroid plexus reverts most, but not all AD-like disturbances.

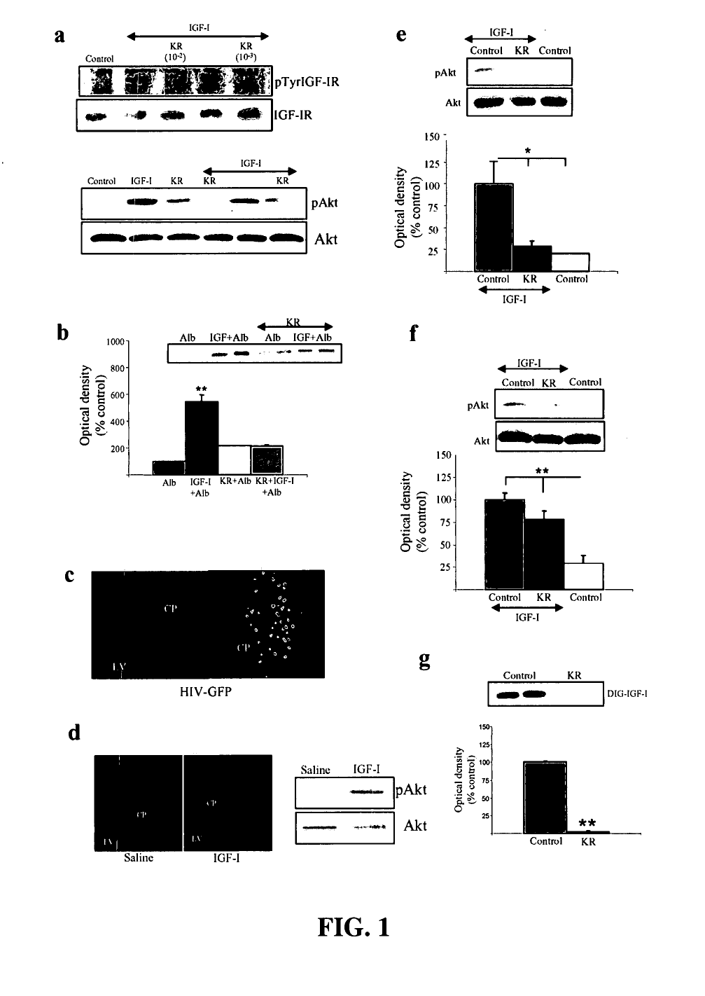

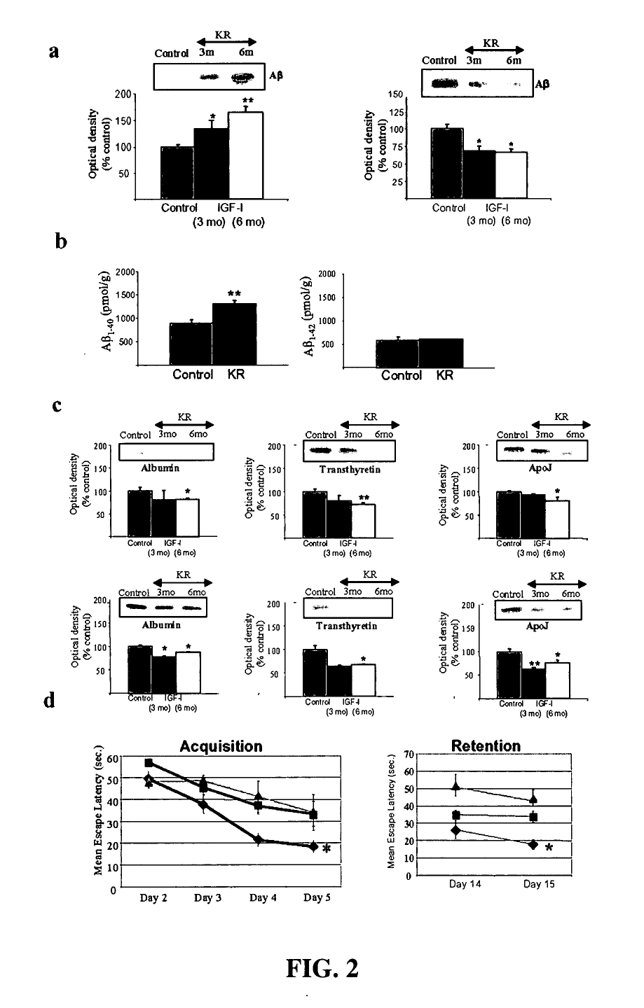

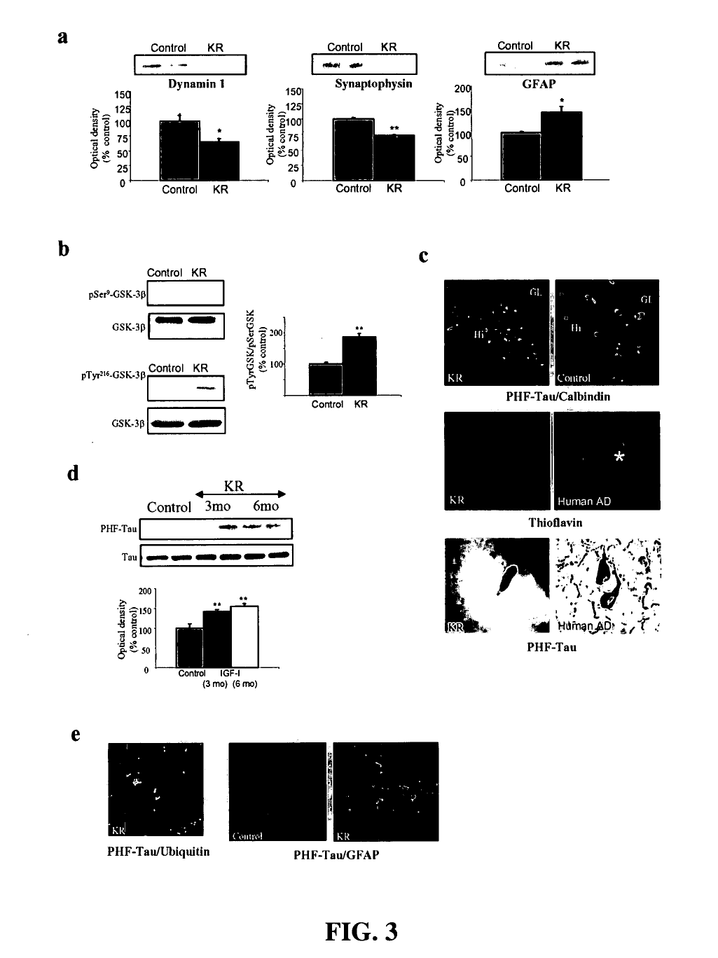

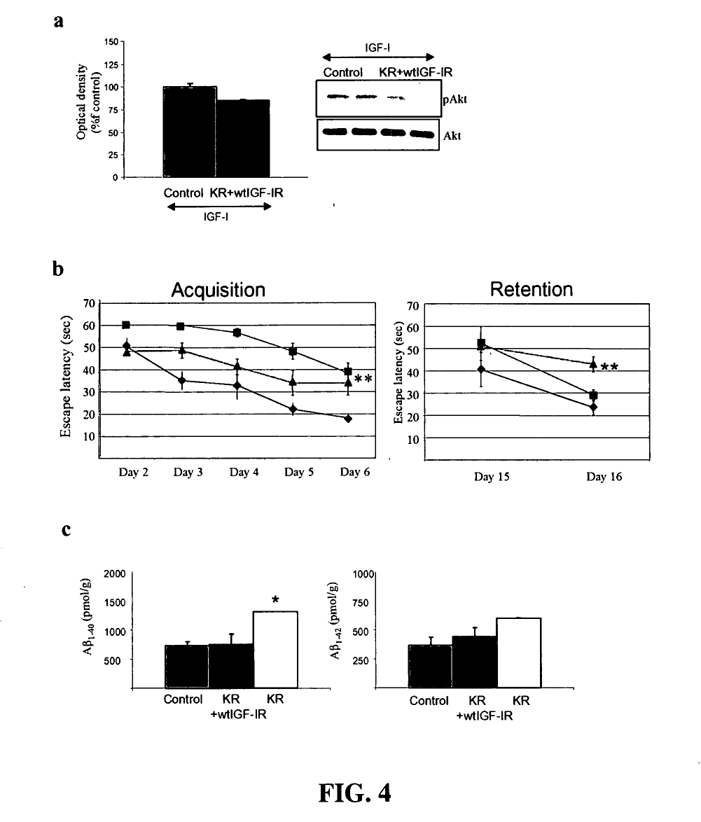

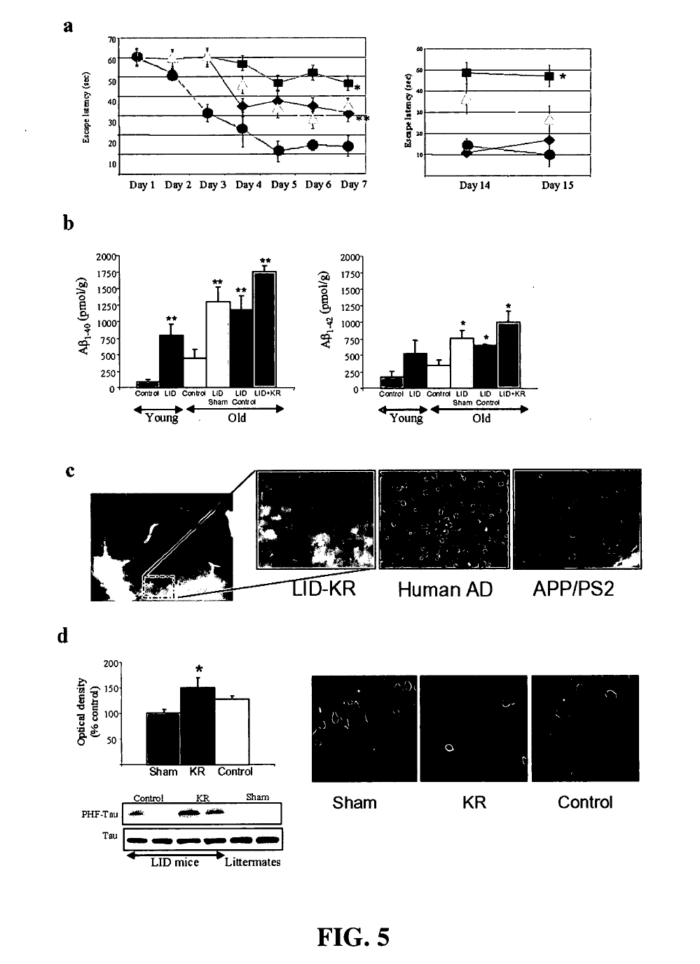

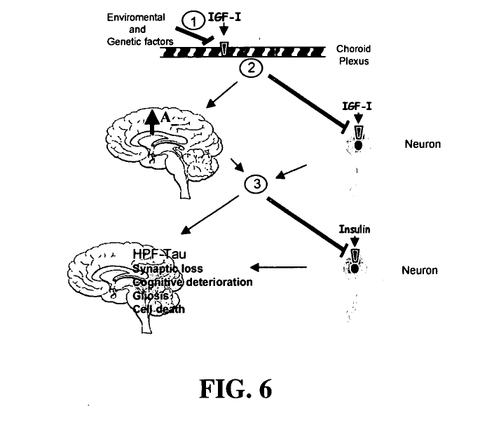

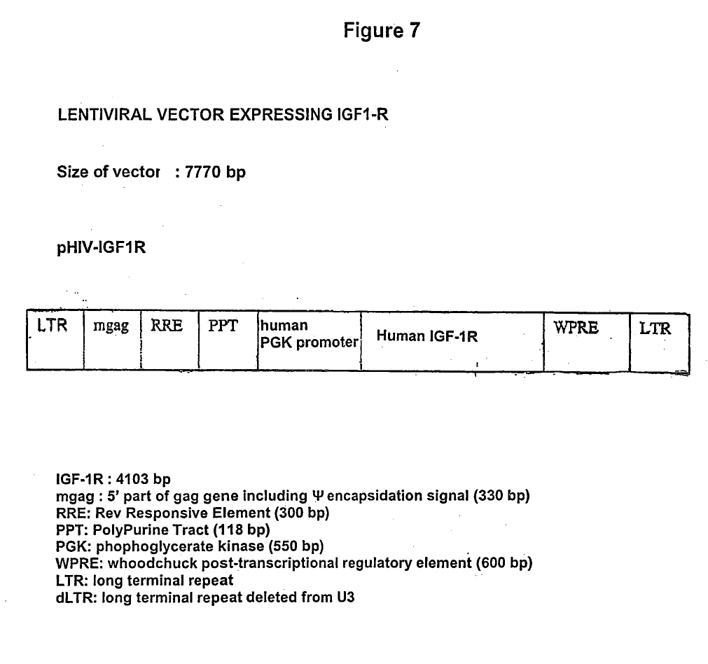

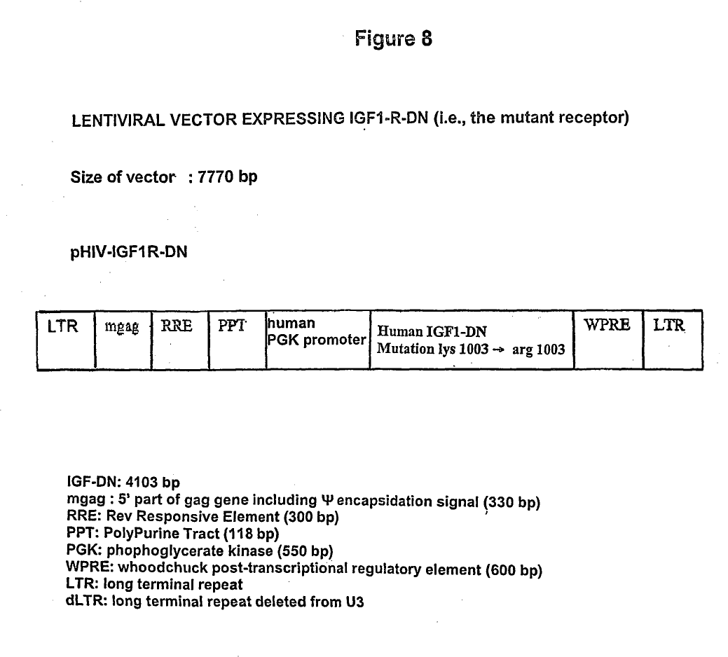

a, Injection of HIV-wild type (wt) IGF-IR to rats that received HIV-KR 3 months before resulted in normalization of choroid plexus responses to IGF-I. After ic injection of IGF-I, KR-wtIGF-IR treated rats show control pAkt levels in choroid plexus (compare this response to that shown by KR rats in FIG. 1e, n=7). b, However, while memory (retention) scores in the water-maze were also normalized after restoring IGF-IR function, learning (acquisition) the location of the platform remained impaired. N=12 controls (rhombs), n=7 KR-wtIGF-IR (squares), and n=6 KR-treated groups (triangles); **p<0.01 vs controls. c, On the contrary, levels of brain Aβ₁₋₄₀ were normalized by wtIGF-IR coexpression with KR. N=7 for all groups; *p<0.01 vs controls.

FIG. 5: Exacerbation of AD-like pathology by KR administration to old mutant mice.

a, Spatial learning and memory in the water maze test is severely impaired in aged LID mice receiving icv KR 3 months before. Note that void vector treated old LID mice show learning impairment similar to age-matched control littermates as compared to young (6 months-old) wild type littermates. N=5 aged-LID-KR injected mice (squares), n=7 aged LID HIV mice (triangles), n=6 aged intact LIDs, n=6 aged littermate mice (rhombs), n=8 young littermate mice (circles), n=6 young LID mice; *p<0.001 vs aged littermates and void-vector LID mice, and **p<0.001 vs young mice. b, Levels of Aβ₁₋₄₀ and of Aβ₁₋₄₂, as determined by ELISA, were not significantly elevated in KR-treated old LID mice as compared to old control LIDs. Note that young LID mice already have high Aβ levels as compared to control littermates and that old (>21 months-old) LIDs show even higher levels. N=; *p<0.05 and **p<0.01 vs respective controls. c, Left: old LID mice treated with KR show scattered small amyloid plaques. Note diffuse amyloid immunostaining in KR animals, absent in controls. Right: amyloid staining in brain sections of LID (left), human AD (center) and APP/PS2 mice (right) reveals the presence of florid plaques only in the two latter. d, Left: Levels of PHF-tau are significantly increased in KR-treated old LID mice. Representative blot and densitometry is shown. n=5 LID-KR; n=7 LID HIV; n=8 littermates (sham); N=; *p<0.05 vs controls. Right: abundant PHF-tau (red) profiles are found in the hippocampus of LID-KR mice as compared to void vector injected LIDs (controls) or littermates (sham). Neurons are stained with βIII tubulin (green). ML, molecular layer.

FIG. 6: Proposed pathogenic processes in sporadic Alzheimer's disease.

1: Although during normal aging there is a gradual decline in IGF-I input³⁷, an abnormally high loss of IGF-I input in the choroid plexus develops in sporadic AD as a result of genotype/phenotype interactions. 2: Consequently, Aβ clearance is compromised and Aβ accumulates in brain. In parallel, neuronal IGF-I input is impaired through reduced entrance of systemic IGF-I (see FIG. 1e), associated to increased neuronal resistance to IGF-I (unpublished observations). 3: Loss of sensitivity of neurons to insulin¹⁹ is brought about by the combined loss of sensitivity to IGF-I²⁴ and excess Aβ⁴⁶. The pathological cascade is initiated: tau-hyperphosphorylation, synaptic derrangement, gliosis, cell death and other characteristic features of AD neuropathology are triggered by the combined action of amyloidosis and loss of IGF-I/insulin input. More work is needed to ascertain the validity of this proposal since the present data do not allow to distinguish between steps 2 and 3.

FIG. 7: Description of Lentiviral vector expressing IGF-1R: pHIV-IGF1R.

The following digestion pattern (expressed in bp) can be found for the plasmid after extraction from bacteria and incubation with the following restriction enzymes.

EcoR1: 5515+4793+541+43

Pst1: 7472+1728+1692

Pvu2: 2942+2519+1748+938+771+767+645+578

Bgl2+Xba1: 4126+3654+2323+682+66+41.

FIG. 8: Description of Lentiviral vector expressing IGF-1R: pHIV-IGF1R-DN.

The following digestion pattern (expressed in bp) can be found for the plasmid after extraction from bacteria and incubation with the following restriction enzymes.

EcoR1: 5515+4793+541+43

Pst1: 7472+1728+1692

Pvu2: 2942+2519+1748+938+771+767+645+578

Bgl2+Xba1: 4128 6+3654+2323+682+66+41.

### SEQUENCING

The plasmid region containing mutation in the transgene (lys 1003 or arg 1003) is the region comprised between bases 7700 and 8100 of pHIV-IGF1-DN. For the deposited strain, this region can be sequenced to confirm viability of the microorganism.

### DETAILED DESCRIPTION OF THE INVENTION

While analyzing the neuroprotective actions of circulating insulin-like growth factor I (IGF-I) in the adult brain, the present inventors have surprisingly found that this pleiotropic peptide regulates brain Aβ clearance. By favoring choroid plexus passage into the brain of Aβ carrier proteins, serum IGF-I controls brain Aβ levels⁴. Together with recent therapeutic strategies unveiling the existence of an “amyloid sink” whereby brain Aβ can be rapidly eliminated⁵, these results (see Example Section) supported the possibility that not only decreased/defective Aβ processing but also abnormal brain Aβ clearance contributes to AD amyloidosis⁶. To assess this notion the inventors have determined whether inhibition of IGF-1-mediated brain Aβ clearance in laboratory rodents originates abnormal accumulation of Aβ in the brains of adult healthy animals. Notably, this is the first report showing that impaired clearance of Aβ produced by blockade of IGF-I receptors in the choroid plexus is associated not only to brain amyloidosis but also to accumulation of hyperphosphorylated tau, cognitive derangement, and other neuropathological changes characteristic of AD.

1. Vectors of the Invention

According to an embodiment of the invention, the present invention is concerned with gene transfer vectors capable of either expressing a dominant negative IGF-I receptor or a functional IGF-I receptor. The gene transfer vectors contemplated by the present invention are preferably derived from HIV or adeno-associated viral (AAV) vectors.

Among those vectors that express a dominant negative IGF-I receptor, the present invention preferably consists of the vector deposited at CNCM on Nov. 10, 2004 under accession number I-3316.

Among those vectors that express a functional IGF-I receptor, the present invention preferably consists of the vector deposited at CNCM on Nov. 10, 2004 under accession number I-3315.

As can be appreciated, supplemental informations concerning the vectors of the invention as well as notions on viral vector in general are recited hereafter.

pHIV-IGF1R deposited under N° CNCM I-3315 is a recombinant plasmid derived from pbr322 encoding the genome of a lentiviral vector which carries a transcription unit having:
the promoter of human phosphoglycerate kinase,
a human cDNA encoding the native form of the receptor for Insulin-Growth factor.

The vector is inserted in E. coli E12 cells which can be cultivated in LB medium with ampicillin. Conditions for seeding are 100 μl in 3 ml LB medium with ampicillin and incubation is carried out at 30° C. under shaking.

The storage conditions are freezing at −80° C. in suspending fluid: ½ bacterial culture (100 μl for 3 ml) and ½ glycerol.

According to the CGG classification the deposited microorganism belongs to Group 2, class 2 and L1 type for confinement.

pHIV-IGF1R-DN deposited under N° CNCM I-3316 has the same characteristics as pHIV-IGF1R except for the human cDNA that it contains which encodes a negative transdominant mutant of the receptor for Insulin-Growth factor according to Fernandez et al 2001. Genes Dev. 15: 1926-1934.

2. Non-Human Animal Disease Model

According to another embodiment, the present invention relates to a non-human animal used as a model for disease where abnormal brain accumulation of β amyloid and/or amyloid plaques are involved, wherein β amyloid clearance from brain is decreased. Such a disease preferably comtemplated by the present invention is Alzheimer's disease. As used herein, the term “non-human animal” refers to any non-human animal which may be suitable for the present invention. Among those non-human animals, rodents such as mice and rats, and primates such as cynomolgus macaques (Macaca fascicularis) are preferred. The cited animals are examples of animals suitable for use as models, i.e., animals suitable for constituting laboratory animals. The invention is especially directed to such laboratory animals, used or intended for use in research or testing.

According to a preferred embodiment, the IGF-IR function of the animal of the invention is impeded in the choroid plexus epithelium. Even more preferably, the IGF-IR function of the animal is impeded by gene transfer into the choroid plexus epithelial cells with a gene transfer vector as defined above which expresses a dominant negative IGF-I receptor. Preferably, such a vector is the one deposited at CNCM on Nov. 10, 2004 under accession number I-3316.

Therefore, the invention relates especially to non-human transgenic animal wherein gene transfer has been carried out in order to impede the IGF-IR function of the original animal. Accordingly, where reference is made in the present application, to non-human animal suitable for use as disease model, it encompasses such transgenic animals. In a preferred embodiment, a non-human animal suitable for use as disease model specifically corresponds to such transgenic animals.

3. Methods of Use

According to another embodiment, the present invention provides a method for screening a molecule for the treatment of diseases where abnormal brain accumulation of β amyloid and/or amyloid plaques are involved wherein said method comprises administering said molecule to an animal as defined above during a time and in an amount sufficient, for the Alzheimer's disease-like disturbances to revert, wherein reversion of Alzheimer's disease-like disturbances is indicative of a molecule for the treatment of diseases where abnormal brain accumulation of β amyloid and/or amyloid plaques are involved.

By the term “treating” is intended, for the purposes of this invention, that the symptoms of the disease be ameliorated or completely eliminated.

The invention also relates to a method for screening a molecule for preventing a disease (including for preventing its symptoms to arise), where said disease (or symptoms) involve abnormal brain accumulation of β amyloid and/or amyloid plaques, wherein said method comprises administering said molecule to an animal as defined above and detecting if Alzheimers disease-like disturbances arrive, wherein where if such disturbances do not appear after a period of observation whereas such disturbances appear in the same type of animal during the same period of observation when said same type of animal has not been received said molecule, the molecule is considered to be a candidate to prevent the disease.

The method of screening according to the invention is a method aiming at determining the effect of a test molecule on disturbances induced by or expressed in Alzheimer's disease-like diseases.

Accordingly, the screening method of the invention encompasses using an animal as defined in the invention, administering the test molecule to said animal, determining the effect of said test molecule on the disturbances of concern and possibly including at some stage sacrifying the animal.

The invention also relates to the use of the animal described according to the invention, as animal model in a screening method for test molecules.

The screening method can comprise, in the frame of the determination of the effect of the test molecule on disturbances of concern, brain imaging (e.g., MRI (Magnetic Resonance Imaging), PET scan (Ponction Emission Tomography scan)) and/or behavioral evolution of the animal model and/or in vitro studies on the effects of said test molecules on samples, especially tissue or cell extracts, obtained from said animal.

According to another embodiment, the present invention provides a method for treating a disease, such as Alzheimer's disease, where abnormal brain accumulation of β amyloid and/or amyloid plaques are involved in a mammal, such as a human, wherein said method comprises administering to said mammal a molecule capable of increasing β amyloid clearance from brain. According to a preferred embodiment, the clearance of β amyloid is increased by increasing the activity of IGF-I receptor in. choroid plexus epithelial cells.

The invention also relates to the use of a test molecule that has shown to improve or revert condition in a patient having Alzheimer's disease-like disturbances in a method of screening of the invention, for the preparation of a drug for the treatment of an Alzheimer or an Alzheimer-like disease.

It will be understood that such a molecule contemplated by the present invention preferably promotes the entrance of a protein acting as a carrier of β amyloid through the choroid plexus into the cerebrospinal fluid. Advantageously, the carrier is chosen from albumin, transthyretin, apolipoprotein J or gelsolin.

According to a preferred embodiment, the molecule which is administered to the animal for increasing said IGF-I receptor activity is a gene transfer vector capable of inducing the expression of IGF-I receptor in target cells, such as one as described above and more preferably, the vector deposited at CNCM on Nov. 10, 2004 under accession number 1-3315.

The molecule to be used in the treating method of the invention is preferably administered to the mammal in conjunction with an acceptable vehicle. As used herein, the expression “an acceptable vehicle” means a vehicle for containing the molecules preferably used by the treating method of the invention that can be administered to a mammal such as a human without adverse effects. Suitable vehicles known in the art include, but are not limited to, gold particles, sterile water, saline, glucose, dextrose, or buffered solutions. Vehicles may include auxiliary agents including, but not limited to, diluents, stabilizers (i.e., sugars and amino acids), preservatives, wetting agents, emulsifying agents, pH buffering agents, viscosity enhancing additives, colors and the like.

The amount of molecules to be administered is preferably a therapeutically effective amount. A therapeutically effective amount of molecules is the amount necessary to allow the same to perform its desired role without causing overly negative effects in the animal to which the molecule is administered. The exact amount of molecules to be administered will vary according to factors such as the type of condition being treated, the mode of administration, as well as the other ingredients jointly administered.

The molecules contemplated by the present invention may be given to a mammal through various routes of administration. For instance, the molecules may be administered in the form of sterile injectable preparations, such as sterile injectable aqueous or oleaginous suspensions. These suspensions may be formulated according to techniques known in the art using suitable dispersing or wetting agents and suspending agents. The sterile injectable preparations may also be sterile injectable solutions or suspensions in non-toxic parenterally-acceptable diluents or solvents. They may be given parenterally, for example intravenously, intradermally, intramuscularly or sub-cutaneously by injection, by infusion or per os. Suitable dosages will vary, depending upon factors such as the amount of the contemplated molecule, the desired effect (short or long term), the route of administration, the age and the weight of the mammal to be treated. Any other methods well known in the art may be used for administering the contemplated molecule.

In a related aspect and according to another embodiment, the present invention is concerned with the use of the nucleotide sequence encoding the receptor of IGF-I for the treatment or prevention of a disease, such as Alzheimer's disease, where abnormal brain accumulation of f amyloid and/or amyloid plaques are involved.

Reference is made to Ebina Y. et al, 1985 (Cell. April, 40(4): 747-58) and Ullrich A. et al (1985 (Nature February 28-March 6, 313 (6005): 756-61) regarding the description of human insulin receptor coding sequence.

The sequence of the human IGF-I is contained as an insert within vector pHIV-IGFIR deposited at the CNCM under N° I-3315.

The invention also relates to the use of a nucleotide sequence encoding a polypeptide having a function analogous to the function of the IGF-I receptor, for the prevention or the treatment of a disease where abnormal brain accumulation of β amyloid and/or amyloid plaques are involved, such a nucleotide sequence encoding a polypeptide which is an active fragment of the IGF-1 receptor.

An “active fragment” means a polypeptide having part of the amino acid sequence of the IGF-1 receptor and which has effect on the regulation of Aβ clearance as disclosed above.

A polypeptide having an analogous function to that of the IGF-1 receptor is a polypeptide similar to said receptor when considering the regulation of Aβ clearance as disclosed above.

The invention also encompasses a therapeutic composition comprising a nucleotide sequence encoding a polypeptide having an analogous function to the function of the IGF-I receptor.

Such a therapeutic composition can comprise a polynucleotide coding for an active fragment of the IGF-1 receptor as described above.

In a particular embodiment, it comprises the pHIV-IGF1R vector.

4. Process and Other Use of the Invention

According to another embodiment, the present invention provides a process for screening an active molecule interacting with the IGF-I receptor comprises administering said molecule to an animal during a time and in an amount sufficient for Alzheimer's disease-like disturbances to be modulated, wherein reversion of Alzheimer's disease-like disturbances is indicative of a molecule that increases IGF-I receptor activity and wherein appearance of Alzheimer's disease-like disturbances is indicative of a molecule that decreases IGF-I receptor activity. Advantegously, reversion of Alzheimer's disease-like disturbances is observed in an animal as defined above.

The present invention will be more readily understood by referring to the following example. This example is illustrative of the wide range of applicability of the present invention and is not intended to limit its scope. Modifications and variations can be made therein without departing from the spirit and scope of the invention. Although any methods and materials similar or equivalent to those described herein can be used in the practice for testing of the present invention, the preferred methods and materials are described.

### EXAMPLE

### Alzheimer's-Like Neuropathology After Blockade of Insulin-Like Growth Factor I Signaling in the Choroid Plexus

Aging, the major risk factor in Alzheimer's disease (AD), is associated to decreased input of insulin-like growth factor I (IGF-I), a purported modulator of brain β amyloid (Aβ) levels. The inventors now present evidence that reduced Aβ clearance due to impaired IGF-I receptor (IGF-IR) function originates not only amyloidosis but also other pathological traits of AD. Specific blockade of the IGF-IR in the choroid plexus, a brain structure involved in Aβ clearance by IGF-I, led to brain amyloidosis, cognitive impairment and hyperphosphorylated tau deposits together with other AD-related disturbances such as gliosis and synaptic protein loss. In old mutant mice with AD-like disturbances linked to abnormally low serum IGF-I levels, IGF-IR blockade in the choroid plexus exacerbated AD-like pathology. These findings shed light into the causes of late-onset Alzheimer's disease suggesting that an abnormal age-associated decline in IGF-I input to the choroid plexus contributes to development of AD in genetically-prone subjects.

Methods

Viral Vectors

Dominant negative (DN) and wild type (wt) IGF-I receptor (IGF-IR) cDNAs were subcloned in the SamI/XbaI site of the HIV-1-phosphoglycerate kinase 1 (PGK) transfer vector⁴⁰. The green fluorescent protein (GFP) cDNA was subcloned in the BamHI/SalI site. The HIV-I-PGK vector bound up in the SamI/XbaI site was used as a control (void vector). The packaging construct and the vesicular stomatitis virus G protein envelope included the pCMVΔR-8.92, pRSV-Rev and pMD.G plasmids⁴¹, respectively. The transfer vector (13 μg), the envelope (3.75 μg), and the packaging plasmids (3.5 μg) were co-transfected with calcium phosphate in 293 T cells (5×10⁶ cells/dish) cultured in Dulbecco's modified Eagle's medium (DMEM, Gibco, USA) with 10% FCS, 1% glutamine and 1% penicillin/streptomycin. Medium was changed 2 hrs prior to transfection and replaced after 24 hrs. Conditioned medium was collected 24 hrs later, cleared (1000 rpm/5 min), and concentrated ≈100 fold (19000 rpm/1.5 hrs). The pellet was re-suspended in phosphate-buffered saline with 1% bovine serum albumin, and the virus stored at −80° C. Viral title was determined by HIV-1 p24 ELISA (Perkin Elmer, USA).

Experimental Design

Wistar rats (5-6 months old, ˜300 g), and liver-IGF-I-deficient (LID) mice (6-21 months old, ˜25-30 g) were from our inbred colony. Animals were used following EEC guidelines. To minimize animal use the inventors initially compared responses of intact (sham) animals with those obtained in void-vector treated animals (see below) and since no differences were appreciated (see for example FIGS. 1d-f) the inventors used only the latter group as controls.

Viral suspensions (140 μg HIV-1 p24 protein/ml, 6 μl/rat and 2 μl/mouse) were stereotaxically injected in each lateral ventricle (rat brain coordinates: 1 posterior from bregma, 1.2 lateral and 4 mm ventral; mouse: 0.6 posterior, 1.1 lateral and 2 mm ventral) with a 10 μl syringe at 1 μl/min. Recombinant IGF-I (GroPep, Australia) was labelled with digoxigenin (DIG, Pierce, USA) as described⁸ and administered as a bolus injection either into the brain parenchyma (1 μg/rat; stereotaxic coordinates: 3.8 posterior from bregma, 2 lateral and 3.2 mm ventral,) or through the carotid artery (10 μg/rat). Cerebrospinal fluid (CSF) was collected under anesthesia from the cisterna magna. Animals were perfused transcardially with saline buffer or 4% paraformaldehyde in 0.1 M phosphate buffer (PB, pH 7.4) for biochemical and immunohistochemical analysis, respectively.

In in vitro studies a double-chamber choroid plexus epithelial cell culture system mimicking the blood-cerebrospinal (CSF) interface was used as described⁴. For viral infection, fresh DMEM containing the virus (≈1 μg/ml) and 8 μg/ml polybrene (Sigma) was added and replaced after 24 hrs. Cells were incubated another 24 hrs and thereafter IGF-I (100 nM) and/or DIG-albumin (1 μg/ml) added to the upper chamber. Lower chamber medium was collected and cells lysed and processed.

Immunoassays

Western-blot (WB) and immunoprecipitation were performed as described⁴². To analyze Aβ deposits, coronal brain sections were serially cut and pre-incubated in 88% formic acid and immunostained, as described⁴. For detection of total Aβ by ELISA, the inventors used the 4G8 antibody (Sigma) in the lower layer and anti-Aβ₁₋₄₀ or anti-Aβ₁₋₄₂ (Calbiochem, USA) in the top layer. To quantify both soluble and insoluble forms of Aβ, samples were extracted with formic acid and assayed as described⁴³. Human AD brain sections were obtained from Novagen (USA) and APP/PS2 mouse brain was a kind gift of H. Loetscher (Hoffman-La Roche, Switzerland). Mouse anti-Aβ (MBL, Japan) that recognizes rodent and human N-terminal Aβ forms, anti-albumin (Bethyl, USA), anti-transthyretin (Santa Cruz, USA), anti-apolipoprotein J (Chemicon, USA), anti-synaptophysin (Sigma), anti-dynamin 1 (Santa Cruz), anti-GFAP (Sigma), anti-calbindin (Swant, Switzerland), anti-βIII-tubulin (Promega, USA), anti-PHF-tau (AT8, Innogenetics, Belgium), anti-ubiquitin (Santa Cruz), anti-pSer⁹ and anti-pTyr²¹⁶ GSK3β (New England Biolabs, USA), anti-pAkt (Cell Signalling, USA) were all used at 1:500-1:1000 dilution. Secondary antibodies were Alexa-coupled (Molecular Probes, USA) or biotinylated (Jackson Immunoresearch, USA).

Behavioral Evaluation

Spatial memory was evaluated with the water maze test⁴⁴ as described in detail elsewhere⁴⁵. Briefly, after a 1 day habituation trial (day 1) in which preferences between tank quadrants were ruled out, for the subsequent 2-5/6 days the animals learned to find a hidden platform (acquisition), followed by one day of probe trial without the platform—in which swimming speed was found to be similar in all groups, and the preference for the platform quadrant evaluated. Nine to ten days later, animals were tested for long-term retention (memory) with the platform placed in the original location. On the last day, a cued version protocol was conducted to rule out possible sensorimotor and motivational differences between experimental groups. Behavioral data were analyzed by ANOVA and Student's t test.

Results

Blockade of IGF-I Signaling in the Choroid Plexus

Expression of a dominant negative (DN) form of the IGF-I receptor impairs IGF-I signaling⁷. Indeed, viral-driven expression of a DN IGF-IR (KR) in choroid plexus epithelial cells abolishes IGF-1-induced phosphorylation of its receptor and its downstream kinase Akt (FIG. 1a). The inventors previously found that IGF-I promotes the entrance of albumin through the choroid plexus into the CSF⁴. When choroid plexus cells are infected with the HIV-KR vector, IGF-I-induced transcytosis of albumin across the epithelial monolayer is inhibited (FIG. 1b). This indicates that blockade of IGF-IR function impairs passage of an Aβ carrier such as albumin through choroid plexus cells. Therefore, the inventors inhibited IGF-I signaling in the choroid plexus in vivo by intraventricular injection of the HIV-KR vector.

Delivery of HIV-GFP into the brain lateral ventricles (icv) resulted in sustained GFP expression in the choroid plexus epithelium of the lateral ventricles and adjacent periventricular cell lining (FIG. 1c). Vessels close to the injection site and the IV ventricle were also labelled (not shown). Using the same icv route, injection of the HIV-KR vector to rats resulted in blockade of IGF-IR function specifically in the choroid plexus, but not in brain parenchyma (FIG. 1d-f). Systemic injection of IGF-I in void vector- or saline-injected rats induces Akt phosphorylation in choroid plexus (FIG. 1d,e). Similarly, injection of IGF-I directly into the brain stimulates Akt phosphorylation in the parenchyma surrounding the injection site (FIG. 1f). However, in KR-injected animals, IGF-I phosphorylates Akt only when injected into the brain (FIG. 1f) but not after intracarotid injection (FIG. 1e), indicating blockade of systemic IGF-I input to the choroid plexus. In addition, passage of blood-borne digoxigenin-labeled IGF-I into the CSF was interrupted, as negligible levels of labeled IGF-I were found in the CSF after intracarotid injection (FIG. 1g). This suggests that intact IGF-IR function at the choroid plexus is required for the translocation of circulating IGF-I into the brain⁸. Altogether these results indicate that viral delivery of a DN IGF-IR into the choroid plexus results in effective blockade of IGF-IR function in this brain structure.

Development of AD-Like Neuropathology After Blockade of IGF-IR Function in the Choroid Plexus.

The inventors hypothesized that blockade of the IGF-IR in the choroid plexus would lead to increased brain Aβ due to reduced entrance of Aβ carriers to the brain⁴. Indeed, after icv injection of HIV-KR, a progressive increase in Aβ₁₋ₓ levels in cortex (FIG. 2a) and hippocampus (not shown), but not in cerebellum (not shown) and a simultaneous decrease in Aβ₁₋ₓ levels in the CSF (FIG. 2a) was found using a pan-specific anti-Aβ. ELISA quantification of Aβ₁₋₄₀ and Aβ₁₋₄₂ showed increased βA₁₋₄₀ in cortex, while βA₁₋₄₂ remained unchanged six months after KR injection (FIG. 2b). No amyloid deposits were found in KR-injected rats using either Aβ₁₋ₓ or Aβ₁₋₄₂-specific antibodies (not shown). A parallel decrease in brain and CSF levels of Aβ carriers such as albumin, apolipoprotein J and transthyretin was also found (FIG. 2c).

Since increased brain Aβ load, even in the absence of amyloid plaques, is associated to impaired cognition in animal models of AD⁹ the inventors determined whether KR-injected rats show learning and memory disturbances. Using the water maze test, an hippocampal-dependent learning paradigm widely used in rodent AD models¹⁰, the inventors found impaired performance in rats as early as 3 months after HIV-KR injection (FIG. 2d). Animals kept for 6 months after HIV-KR have similar cognitive perturbances (FIG. 2d). A decrease in the synaptic vesicle proteins synaptophysin and dynamin 1 is found in AD, a deficit that has been associated to cognitive loss^{11,12}. After KR injection both proteins are decreased (FIG. 3a) while GFAP, a cytoskeletal marker of gliosis associated to neuronal damage in AD¹¹, was elevated (FIGS. 3a,d).

Although amyloidosis is not always associated to the appearance of hyperphosphorylated tau (PHF-tau), the inventors found that 3 months after KR injection, when the animals have amyloidosis, they also have increased levels of PHF-tau. In addition, an increased pTyr²¹⁶GSK-3β (active form)/pSer⁹GSK-3β (inactive form) ratio in the brain of KR-injected rats (FIG. 3b) suggested increased activity of this tau-kinase¹³, which agrees with appearance of intracellular deposits of PHF-tau in neurons (FIG. 3c) and glial cells (FIG. 3d, right panels). Using the AT8 antibody that recognizes PHF-tau in both pre-tangles and tangles¹⁴, intracellular deposits of PHF-tau and increased PHF-tau levels were observed in KR-rats (FIG. 3c). Comparison of KR rats with human AD suggested that intracellular PHF-tau deposits in the former correspond mostly to pre-tangles. Thus, thioflavin-S⁺ and PHF-tau⁺ tangle profiles were observed in human AD but not in KR rat brains (FIG. 3c, middle and lower left panels). PHF-tau deposits associated to ubiquitin and were surrounded by reactive glia (FIG. 3d). Robust PHF-tau staining was also observed in the choroid plexus of KR rats (not shown).

The inventors next restored IGF-IR function in the choroid plexus of rats injected with HIV-KR 3 months before by icv administration of HIV-wtIGF-IR. Animals were evaluated 3 months later to allow for IGF-IR functional recovery; i.e.: 6 months after the initial HIV-KR injection. Following restoration of IGF-IR signaling in the choroid plexus, as determined by normal levels of pAkt in the choroid plexus after intracarotid IGF-I (FIG. 4a), almost full recovery of brain function was achieved. Except for impaired learning (acquisition) in the water-maze (FIG. 4b) all other AD-like disturbances were reverted, including memory loss (FIG. 4, Table 1).

Blockade of IGF-IR Function in the Choroid Plexus Exacerbates AD-Like Traits in Old Mutant Mice

Normal adult KR-treated rats do not develop plaques even though they have high brain Aβ₁₋₄₀ levels. Absence of plaques may be because KR rats have unaltered levels of Aβ₁₋₄₂, the preferred plaque-forming Aβ peptide¹⁵ or because age-related changes in the brain may be necessary to develop plaques. However, it is well known that while aging rodents show a greater incidence of impaired cognition and increased brain Aβ levels, they do not develop Aβ plaques^{16,17}. Despite the latter, the inventors treated aged mutant LID mice¹⁸ with the KR vector. These mice have high brain levels of both Aβ₁₋₄₀ and Aβ₁₋₄₂⁴, and show other age-related changes earlier in life, including low serum IGF-I and insulin resistance¹⁸ that may contribute to AD-like amyloidosis in the brains. With this animal model the inventors aimed to better reproduce the conditions found in the aged human brain to gain further insight into the process underlying AD-like changes after blockade of choroid plexus IGF-IR.

Three months after KR injection, LID mice show disturbed water-maze learning and memory as compared to void-vector injected old LID mice (FIG. 5a). Significantly, aged control LIDs, as age-matched littermates, are already cognitively deteriorated when compared to young littermates (FIG. 5a). Therefore, blockade of IGF-IR function produces further cognitive loss. In addition, KR-injected old LID mice show increases in brain Aβ₁₋₄₀ and Aβ₁₋₄₂, as determined by ELISA, but not significantly different from control old LID mice that had already high levels of both (FIG. 5b). LID-KR injected mice have small insoluble (formic-acid resistant) amyloid plaques that are also occasionally found in old, but not young control LIDs (FIG. 5c). These deposits represent diffuse amyloid plaques²⁰ since they do not Stain with Congo red or thioflavin-S as human AD plaques (not shown) and do not have the compact appearance of human AD or mutant mice amyloid plaques (FIG. 5c). Similarly to changes found in adult rats treated with the KR vector, old LID mice presented HPF-tau deposits and higher levels of HPF-tau 3 months after KR injection (FIG. 5d). Slightly higher GFAP levels (already significantly increased in control LID mice⁴), and synaptic protein loss were also found after KR injection in old LID mice (Table 2).

Discussion

These results indicate that IGF-IR blockade in the choroid plexus triggers AD-like disturbances in rodents including cognitive impairment, amyloidosis, hyperphosphorylated tau deposits, synaptic vesicle protein loss and gliosis. Most of these disturbances could be rescued by reverting IGF-IR blockade, although learning remained impaired. On the contrary, AD-like traits, in particular cognitive loss, were exacerbated when IGF-IR blockade was elicited in aged animals with lower than normal serum IGF-I levels. Although a general decrease in IGF-IR function is associated to normal aging²¹, these results suggest that loss of IGF-IR signaling in the choroid plexus may be, linked to late-onset Alzheimer's disease²². While the causes of familial forms of AD-encompassing merely 5% of the cases¹, are slowly being unveiled, the etiology of sporadic AD is not established. Therefore, insight into mechanisms of reduced sensitivity to IGF-I at the choroid plexus may help unveil the origin of sporadic AD. For instance, risk factors associated to AD may contribute to a greater loss of IGF-IR function in the choroid plexus in affected individuals. Late-onset AD patients could present loss of sensitivity to the Aβ reducing effects of IGF-I. Intriguingly, slightly elevated serum IGF-I levels were found in a pilot study of sporadic AD patients²³, a condition compatible with loss of sensitivity to IGF-1²⁴.

Animal models of AD have successfully recreated several, but not all the major neuropathological changes of this human disease^{25,26}. Most have been developed through genetic manipulation of candidate disease-associated human proteins that usually include widespread expression of the mutated protein²⁷. Recently, a combined transgenic approach targeting three different AD-related proteins led to a mouse model that recapitulates the three main characteristics of AD: cognitive loss, amyloid plaques and tangles²⁸. In the present model, blockade of IGF-IR function specifically in the choroid plexus originates the majority of changes seen in AD brains except amyloid plaques and tangles. For instance, AD-like changes in our model include a reduction in dynamin 1 levels, also found in AD brains but not in animal models of AD amyloidosis¹², reduced CSF tranthyretin levels, also seen in AD²⁹, but not reported in animal models of the disease, or choroid plexus tauopathy, a common finding in AD patients³⁰.

However, the lack of amyloid plaques and neurofibrillary tangles in the present model may question a significant pathogenic role of choroid plexus IGF-IR dysfunction in AD. It seems likely that additional factors, not reproduced in the present rodent model, are required to develop plaques and tangles. This is not surprising since under normal conditions rodents do not develop plaques or tangles³¹, unless forced to express mutant APP or tau (but see refs.^{32,33}). A shorter life-span, or structural differences in APP³¹ may account for this inter-species difference. In addition, while the largest amyloidosis the inventors observed was a mere ≈14-fold increase in total Aβ₁₋₄₀ after IGF-IR blockade in old LID mice, the aging human AD brain can produce substantial amounts of amyloid (well over 300-fold¹⁵), an effect that can be reproduced in rodent models of amyloidosis²⁷. Therefore, under proper experimental settings the rodent brain do produce plaques and tangles²⁸. Thus, the inventors hypothesize that the model recreates, within a rodent context, the initial stages of human sporadic Alzheimer's disease, when plaques and tangles are not yet formed.

Alternatively, development of plaques and tangles may be part of the pathological cascade idiosyncratic to humans (not reproducible in the normal rodent brain), and unrelated to the pathogenesis of the disease. As a matter of fact, the contribution of plaques and tangles to cognitive loss, the clinically relevant aspect of AD, is questionable. In agreement with the present findings, cognitive impairment may develop with brain amyloidosis without plaques³⁴. Similarly, high levels of HPF-tau without tangle formation are also associated to cognitive loss³. Therefore, while current animal models of AD tend to emphasize the occurrence of plaques and tangles, the fact is that cognitive impairment does not depend in either one. Furthermore, amyloid plaques are not always associated to cognitive deterioration³⁶. At any rate, the present results reinforce the emerging notion that high amyloid and/or HPF-tau are sufficient to produce cognitive derangement.

The inventors previously found that serum IGF-I promotes brain Aβ clearance⁴. In response to blood-borne IGF-1, the choroid plexus epithelium translocates Aβ carrier proteins from the blood into the CSF. While low serum IGF-I levels, together with loss of sensitivity to IGF-I associated to aging³⁷ will affect target cells throughout the body, the inventors recently proposed that reduced IGF-I signaling specifically at the choroid plexus would interfere with Aβ clearances. Indeed, the increase in brain Aβ together with decreased levels of Aβ carriers that we now found after IGF-IR blockade, support this notion. Notably, interruption of IGF-I signaling at the choroid plexus elicited not only amyloidosis but also other characteristic disturbances associated to AD. The amyloid hypothesis of AD favors accumulation of amyloid as the primary pathogenic events. However, the factors contributing to amyloid deposition in sporadic AD are not known. Both impaired degradation of Aβ and/or clearance, or excess production could be responsible. The present results indicate that Aβ accumulation due to impaired clearance may be sufficient to initiate the pathological cascade. In this sense, the primary disturbance would be loss of function of the IGF-IR at the choroid plexus, which in turn may originate the pathological cascade due to excess amyloid². Therefore, by placing loss of IGF-I input upstream of amyloidosis the inventors can easily reconcile their observations with current pathogenic concepts of late-onset AD (FIG. 6).

Nevertheless, the inventors' observations leave open several issues. The inventors cannot yet determine the hierarchical relationship between tauopathy and amyloidosis because in their study accumulation of PHF-tau coincided in time with high levels of Aβ. In addition, the inventors observed increases in Aβ₁₋₄₀ but not in Aβ₁₋₄₂ in KR-injected rats. This agrees with the observation that the greatest increase in human AD is in Aβ₁₋₄₀ but Aβ₁₋₄₂ also increases in humans³⁸. Since increases in AD, 42 are found in mutant LID mice⁴, life-long exposure to low IGF-I input may be necessary for Aβ₁₋₄₂ to accumulate in rodent brain within a wild type background of APP and APP-processing proteins. Finally, while reversal of IGF-IR blockade in the choroid plexus rescued most AD-like changes, the animals still have deranged learning. Therefore, AD-like changes following IGF-IR blockade may compromise learning abilities even after been reverted, a finding that differs from that observed in current models of AD amyloidosis where reduction of amyloid load usually accompanies cognitive recovery³⁶.

In conclusion, by specifically blocking IGF-IR function in the choroid plexus (as opposed to the general loss of IGF-I input associated to aging³) the inventors have unveiled a mechanism whereby pathognomonic signs of AD develop. This occurs within a wild type background of AD-relevant proteins such as APP or tau, resembling more closely sporadic forms of human AD. The non-human model of the present invention is relevant for analysis of pathogenic pathways in AD, definition of new therapeutic targets and drug testing. In this regard, blockade of IGF-IR in animal models of AD and AD-related pathways may help gain insight into the interactions between pathogenic routes, risk factors and secondary disturbances. Because the inventors' observations favor that late-onset AD is related to age-dependent reduction in Aβ clearance, drug development may be aimed towards its enhancement. Based on the success in developing insulin sensitizers for type 2 diabetes, enhancement of sensitivity to IGF-1 in AD patients may be already within reach since the two hormones share common intracellular pathways.

TABLE 1
Restoring IGF-IR function in the choroid plexus of KR-injected rats
with HIV-wtIGF-IR reverts AD-like changes in brain levels of various
AD-related proteins

| | KR | KR + wt IGF-IR |
|---|---|---|
| AD-related proteins | (% Control) | (% Control) |
| Aβ₁₋ₓ | 179 ± 8* | 101 ± 30 |
| PHF-Tau | 154 ± 7** | 99 ± 5 |
| GFAP | 198 ± 29* | 119 ± 11 |
| Synaptophysin | 72 ± 1** | 108 ± 4 |
| Dynamin 1 | 64 ± 5 | 102 ± 5 |

Protein levels were determined by WB and quantified by densitometry. Control, void-vector injected rats, n = 7; KR, n = 7; KR + wtIGF-IR n = 7.
*p < 0.05 and
**p < 0.01 vs control.

TABLE 2
Blockade of IGF-IR in choroid plexus of serum IGF-I deficient (LID)
old mice results in AD-like changes in various AD-related proteins.

| | LID-KR |
|---|---|
| AD-related proteins | (% Control) |
| GFAP | 112 ± 2* |
| Synaptophysin | 50 ± 2** |
| Dynamin 1 | 85 ± 1.5** |

Protein levels were determined by WB and quantified by densitometry. Control, void-vector injected old LID mice, n = 5; LID-KR, n = 5.
*p < 0.05 and
**p < 0.01 vs control.

### REFERENCE LIST

1. Mayeux, R. Epidemiology of neurodegeneration. Annu. Rev. Neurosci. 26, 81-104 (2003).
2. Selkoe, D. J. Clearing the Brain's Amyloid Cobwebs. Neuron 32, 177-180 (2001).
3. Golde, T. E. Alzheimer disease therapy: can the amyloid cascade be halted? J. Clin Invest 111, 11-18 (2003).
4. Carro, E., Trejo, J. L., Gomez-lsla, T., LeRoith, D. & Torres-Aleman, I. Serum insulin-like growth factor I regulates brain amyloid-beta levels. Nat. Med. 8, 1390-1397 (2002).
5. DeMattos, R. B. et al. Peripheral anti-A beta antibody alters CNS and plasma A beta clearance and decreases brain A beta burden in a mouse model of Alzheimer's disease. Proc. Natl. Acad. Sci. U.S. A 98, 8850-8855 (2001).
6. Banks, W. A., Robinson, S. M., Verma, S. & Morley, J. E. Efflux of human and mouse amyloid beta proteins 1-40 and 1-42 from brain: impairment in a mouse model of Alzheimer's disease. Neuroscience 121, 487-492 (2003).
7. Fernaridez, A. M. et al. Functional inactivation of the IGF-I and insulin receptors in skeletal muscle causes type 2 diabetes. Genes Dev. 15, 1926-1934 (2001).
8. Carro, E., Nunez, A., Busiguina, S. & Torres-Aleman, I. Circulating insulin-like growth factor I mediates effects of exercise on the brain. J. Neurosci. 20, 2926-2933 (2000).
9. Holcomb, L. A. et al. Behavioral changes in transgenic mice expressing both amyloid precursor protein and presenilin-1 mutations: lack of association with amyloid deposits. Behav. Genet. 29, 177-185 (1999).
10. D'Hooge, R. & De Deyn, P. P. Applications of the Morris water maze in the study of learning and memory. Brain Res. Brain Res. Rev. 36, 60-90 (2001).
11. Ingelsson, M. et al. Early Abeta accumulation and progressive synaptic loss, gliosis, and tangle formation in AD brain. Neurology 62, 925-931 (2004).
12. Yao, P. J. et al. Defects in expression of genes related to synaptic vesicle trafficking in frontal cortex of Alzheimer's disease. Neurobiol. Dis. 12, 97-109 (2003).
13. Liu, S. J. et al. Overactivation of glycogen synthase kinase-3 by inhibition of phosphoinositol-3 kinase and protein kinase C leads to hyperphosphorylation of tau and impairment of spatial memory. J. Neurochem. 87, 1333-1344 (2003).
14. Lauckner, J., Frey, P. & Geula, C. Comparative distribution of tau phosphorylated at Ser262 in pre-tangles and tangles. Neurobiol. Aging 24, 767-776 (2003).
15. Gravina, S. A. et al. Amyloid beta protein (A beta) in Alzheimer's disease brain. Biochemical and immunocytochemical analysis with antibodies specific for forms ending at A beta 40 or A beta 42(43). J. Biol. Chem. 270, 7013-7016 (1995).
16. van der Staay, F. J. Assessment of age-associated cognitive deficits in rats: a tricky business. Neurosci. Biobehav. Rev. 26, 753-759 (2002).
17. Vaucher, E. et al. Amyloid beta peptide levels and its effects on hippocampal acetylcholine release in aged, cognitively-impaired and -unimpaired rats. J. Chem. Neuroanat 21, 323-329 (2001).
18. Yakar, S. et al. Normal growth and development in the absence of hepatic insulin-like growth factor I. Proc. Natl. Acad. Sci. U.S.A 96, 7324-7329 (1999).
19. Gasparini, L. & Xu, H. Potential roles of insulin and IGF-1 in Alzheimer's disease. Trends Neurosci. 26, 404-406 (2003).
20. Ikeda, S., Allsop, b. & Glenner, G. G. Morphology and distribution of plaque and related deposits in the brains of Alzheimer's disease and control cases. An immunohistochemical study using amyloid beta-protein antibody. Lab Invest 60, 113-122 (1989).
21. Lieberman, S. A., Mitchell, A. M., Marcus, R., Hintz, R. L. & Hoffman, A. R. The insulin-like growth factor I generation test: resistance to growth hormone with aging and estrogen replacement therapy. Horm. Metab Res. 26, 229-233 (1994).
22. Carro, E. & Torres-Aleman, I. The role of insulin and insulin-like growth factor I in the molecular and cellular mechanisms underlying the pathology of Alzheimer's disease. Eur. J. Pharmacol. 490, 127-133 (2004).
23. Tham, A. et al. Insulin-like growth factors and insulin-like growth factor binding proteins in cerebrospinal fluid and serum of patients with dementia of the Alzheimer type. J. Neural Transm. Park Dis. Dement. Sect. 5, 165-δ 76 (1993).
24. Jain, S., Golde, D. W., Bailey, R. & Geffner, M. E. Insulin-like growth factor-I resistance. Endocr. Rev. 19, 625-646 (1998).
25. Hock, B. J., Jr. & Lamb, B. T. Transgenic mouse models of Alzheimer's disease. Trends Genet. 17, S7-12 (2001).
26. Schwab, C., Hosokawa, M. & McGeer, P. L. Transgenic mice overexpressing amyloid beta protein are an incomplete model of Alzheimer disease. Exp. Neurol. 188, 52-64 (2004).
27. Kawarabayashi, T. et al. Age-dependent changes in brain, CSF, and plasma amyloid (beta) protein in the Tg2576 transgenic mouse model of Alzheimer's disease. J. Neurosci. 21, 372-381 (2001).
28. Oddo, S. et al. Triple-transgenic model of Alzheimer's disease with plaques and tangles: intracellular Abeta and synaptic dysfunction. Neuron 39, 409-421 (2003).
29. Serot, J. M., Bene, M. C. & Faure, G. C. Choroid plexus, ageing of the brain, and Alzheimer's disease. Front Biosci. 8, S515-S521 (2003).
30. Miklossy, J. et al. Curly fiber and tangle-like inclusions in the ependyma and choroid plexus—a pathogenetic relationship with the cortical Alzheimer-type changes? J. Neuropathol. Exp. Neurol. 57, 1202-1212 (1998).
31. De Strooper, B. et al. Production of intracellular amyloid-containing fragments in hippocampal neurons expressing human amyloid precursor protein and protection against amyloidogenesis by subtle amino acid substitutions in the rodent sequence. EMBO J. 14, 4932-4938 (1995).
32. Capsoni, S. et al. Alzheimer-like neurodegeneration in aged antinerve growth factor transgenic mice. Proc. Natl. Acad. Sci. U.S.A 97, 6826-6831 (2000).
33. Little, C. S., Hammond, C. J., Maclntyre, A., Balin, B. J. & Appelt, D. M. Chiamydia pneumoniae induces Alzheimer-like amyloid plaques in brains of BALB/c mice. Neurobiol. Aging 25, 419-429 (2004).
34. Kumar-Singh, S. et al. Behavioral disturbances without amyloid deposits: in mice overexpressing human amyloid precursor protein with Flemish (A692G) or Dutch (E693Q) mutation. Neurobiol. Dis. 7, 9-22 (2000).
35. Avila, J., Lucas, J. J., Perez, M. & Hernandez, F. Role of tau protein in both physiological and pathological conditions. Physiol Rev. 84, 361-384 (2004).
36. Savonenko, A. V., Xu, G. M., Price, D. L., Borchelt, D. R. & Markowska, A. L. Normal cognitive behavior in two distinct congenic lines of transgenic mice hyperexpressing mutant APP SWE. Neurobiol. Dis. 12, 194-211 (2003).
37. Arvat, E., Broglio, F. & Ghigo, E. Insulin-Like growth factor I: implications in aging. Drugs Aging 16, 29-40 (2000).
38. Wang, J., Dickson, D. W., Trojanowski, J. Q. & Lee, V. M. The levels of soluble versus insoluble brain Abeta distinguish Alzheimer's disease from normal and pathologic aging. Exp. Neurol. 158, 328-337 (1999).
39. Younkin, S. G. Amyloid beta vaccination: reduced plaques and improved cognition. Nat. Med. 7, 18-19 (2001).
40. Naldini, L. et al. In vivo gene delivery and stable transduction of nondividing cells by a lentiviral vector. Science 272, 263-267 (1996).
41. Desmaris, N. et al. Production and neurotropism of lentivirus vectors pseudotyped with lyssavirus envelope glycoproteins. Mol. Ther. 4, 149-156 (2001).
42. Pons, S. & Torres-Aleman, I. Insulin-like Growth Factor-I Stimulates Dephosphorylation of Ikappa B through the Serine Phosphatase Calcineurin (Protein Phosphatase 2B). J. Biol. Chem. 275, 38620-38625 (2000).
43. Suzuki, N. et al. An increased percentage of long amyloid beta protein secreted by familial amyloid beta protein precursor (beta APP717) mutants. Science 264, 1336-1340 (1994).
44. Morris, R. Developments of a water-maze procedure for studying spatial learning in the rat. J. Neurosci. Methods 11, 47-60 (1984).
45. Carro, E., Trejo, J. L., Busiguina, S. & Torres-Aleman, I. Circulating insulin-like growth factor I mediates the protective effects of physical exercise against brain insults of different etiology and anatomy. J. Neurosci. 21, 5678-5684 (2001).
46. Xie, L. et al. Alzheimer's beta-amyloid peptides compete for insulin binding to the insulin receptor. J. Neurosci. 22, RC221 (2002).

1. A non-human animal used as a model for disease where abnormal brain accumulation of β amyloid and/or amyloid plaques are involved, wherein β amyloid clearance from brain is decreased.

2. The non-human animal of claim 1, wherein the IGF-IR function of said animal is impeded in the choroid plexus epithelium.

3. The non-human animal of claim 2, wherein the IGF-IR function of said animal is impeded by gene transfer into the choroid plexus epithelial cells with a gene transfer vector expressing a dominant negative IGF-I receptor.

4. The non-human animal of claim 3, wherein said gene transfer vector is derived from HIV or AAV

5. The non human animal of claim 4, wherein said vector was deposited at CNCM on Nov. 10, 2004 under accession number I-3316

6. The non-human animal of claim 1, wherein said disease is Alzheimer's disease.

7. A method for screening a molecule for the treatment of diseases where abnormal brain accumulation of β amyloid and/or amyloid plaques are involved wherein said method comprises administering said molecule to an animal according to claim 1 during a time and in an amount sufficient for the Alzheimer's disease-like disturbances to revert, wherein reversion of Alzheimer's disease-like disturbances is indicative of a molecule for the treatment of diseases where abnormal brain accumulation of β amyloid and/or amyloid plaques are involved.

8. The method of claim 7, wherein said disease is Alzheimer's disease.

9. A method for screening a molecule for the prevention of diseases where abnormal brain accumulation of β amyloid and/or amyloid plaques are involved wherein said method comprises administering said molecule to an animal according to claim 1 and comparing the occurrence of Alzheimer's disease-like in said animal and the occurrence of such Alzheimer's disease-like in the same type of animal which has not received said molecule.

10. A method for treating or preventing a disease where abnormal brain accumulation of β amyloid and/or amyloid plaques are involved in a mammal, wherein said method comprises administering to said mammal a molecule capable of increasing amyloid clearance from brain.

11. The method of claim 10, wherein said molecule promotes the entrance of a protein acting as a carrier of β amyloid through the choroid plexus into the cerebrospinal fluid.

12. The method of claim 11, wherein said carrier is albumin.

13. The method of claim 11, wherein said carrier is transthyretin.

14. The method of claim 11, wherein said carrier is apolipoprotein J.

15. The method of claim 11, wherein said carrier is gelsolin.

16. The method of claim 10, wherein the clearance of β amyloid is increased by increasing the activity of IGF-I receptor in choroid plexus epithelial cells.

17. The method of claim 16, wherein the molecule which is administered to the animal for increasing said IGF-I receptor activity is a gene transfer vector capable of inducing the expression of IGF-I receptor in target cells.

18. The method of claim 17, wherein said gene transfer vector is derived from HIV or AAV.

19. The method of claim 18, wherein said vector was deposited at CNCM on Nov. 10, 2004 under accession number I-3315.

20. A gene transfer vector capable of expressing a dominant negative IGF-I receptor deposited at CNCM on Nov. 10, 2004 under accession number I-3316.

21. A gene transfer vector capable of expressing a functional IGF-I receptor deposited at CNCM on Nov. 10, 2004 under accession number I-3315.

22. A process for screening an active molecule interacting with the IGF-I receptor comprises administering said molecule to an animal during a time and in an amount sufficient for Alzheimer's disease-like disturbances to be modulated, wherein reversion of Alzheimer's disease-like disturbances is indicative of a molecule that increases IGF-I receptor activity and wherein appearance of Alzheimer's disease-like disturbances is indicative of a molecule that decreases IGF-I receptor activity.

23. The process of claim 22, wherein reversion of Alzheimer's disease-like disturbances is observed in an animal according to claim 1.

24. Use of the nucleotide sequence encoding the receptor of IGF-I for the prevention or treatment of a disease where abnormal brain accumulation of β amyloid and/or amyloid plaques are involved.

25. The use of claim 24, wherein said disease is Alzheimer's disease.

26. Use of a nucleotide sequence encoding a polypeptide having a function analogous to the function of the IGF-I receptor, for the prevention or the treatment of a disease where abnormal brain accumulation of, amyloid and/or amyloid plaques are involved.

27. Use according to claim 26, wherein the nucleotide sequence encodes an active fragment of the IGF-I receptor.

28. A therapeutic composition comprising a nucleotide sequence encoding a polypeptide having an analogous function to the function of the IGF-I receptor.

29. A therapeutic composition according to claim 28, wherein the nucleotide sequence encodes an active fragment of the IGF-I receptor.

30. A therapeutic composition which comprises the pHIV-IGF1R vector.
